(19) United States
(12) Patent Application Publication
(10) Pub. No.: US 2016/0031800 A1
(43) Pub. Date: Feb. 4, 2016

(54) SYNTHESIS OF CHIRAL KYNURENINE COMPOUNDS AND INTERMEDIATES

(71) Applicant: VISTAGEN THERAPEUTICS, INC., South San Francisco, CA (US)

(72) Inventors: Alexander TRETYAKOV, Devens, ME (US); Keith E. DROUET, San Diego, CA (US); William SANDERS, Madison, WI (US)

(21) Appl. No.: 14/775,268

(22) PCT Filed: Mar. 14, 2014
(86) PCT No.: PCT/US14/27922

§ 371 Date: Sep. 11, 2015

Related U.S. Application Data
(60) Provisional application No. 61/785,815, filed on Mar. 14, 2013.

(51) Int. Cl. C07C 229/42; C07C 227/20; C07D 209/42

(52) U.S. Cl. CPC C07C 229/42; C07D 209/42; C07C 227/20

(57) ABSTRACT

Provided are methods for the synthesis of compounds including chiral kynurenine compounds, intermediates useful for the synthesis thereof, and related compounds. For example, methods are provided for the synthesis of L-4-chlorokynurenine.

### CROSS REFERENCE TO RELATED APPLICATION

This application claims priority to U.S. Provisional Patent Application No. 61/785,815, filed Mar. 14, 2013, the disclosure of which is hereby incorporated by reference in its entirety.

### TECHNICAL FIELD

The present invention relates to methods for synthesizing compounds, including chiral kynurenine compounds, intermediates in the synthesis thereof, and related compounds.

### BACKGROUND

Kynurenic acid is a metabolically related brain constituent with anticonvulsant and neuroprotective properties (Stone, T. W.; Pharmacol. Rev. 1993, 45, 309-379). The biological activities of various derivatives of kynurenic acid and their kynurenine precursors have been studied (Camacho, E. et al. J. Med. Chem. 2002, 45, 263-274; Varasi, M. et al. Eur. J. Med. Chem. 1996, 31, 11-21; Salituro, F. G. et al. J. Med. Chem. 1994, 37, 334-336). Kynurenine compounds are converted to kynurenic acids in vivo. U.S. Pat. No. 5,547,991 to Merrell Pharmaceuticals, Inc. describes methods of making 4,6-disubstituted tryptophan derivatives and their use as N-methyl-D-aspartate (NMDA) antagonists.

An enantioselective synthesis of L-4-chlorokynurenine described by Salituro et al. was used for the synthesis of gram quantities of L-4-chlorokynurenine (Salituro, F. G. et al. J. Med. Chem. 1994, 37, 334-336). This synthesis was not practical for scale up on a commercial manufacturing scale due to the use of reagents such as trimethyl tin chloride, sodium hydride, and tert-butyllithium and the poor availability of certain building blocks.

A racemic synthesis of 4-chlorokynurenine was reported in Varasai et al. Eur. J. Med. Chem. 1996, 31, 11-21. However, experiments for the separation of the enantiomers by crystallization of diastereomeric salts were not successful, nor was preparative high-performance liquid chromatography (HPLC) substantially successful, due to low solubility.

There is a need for a convenient synthesis of synthesizing compounds, including chiral kynurenines, intermediates in the synthesis thereof, and related compounds using commercially available reagents that does not require the use of toxic or highly reactive reagents or extensive purification techniques. There is a need for syntheses suitable for large-scale manufacture and that can produce compounds, including chiral kynurenines, intermediates in the synthesis thereof, and related compounds in high chemical purity and high chiral purity.

All publications, patents, and patent applications cited herein are hereby incorporated by reference in their entirety for all purposes.

### SUMMARY

Provided are methods for the synthesis of compounds, including chiral kynurenine compounds, intermediates in the synthesis thereof, and related compounds. In a specific embodiment, methods are provided for the synthesis of L-4-chlorokynurenine. In certain embodiments, the syntheses advantageously use commercially available reagents and avoid the use of toxic or highly reactive reagents or extensive purification techniques. In certain embodiments, syntheses are provided that are suitable for large-scale manufacture and suitable for producing the chiral kynurenines in high chemical purity and high chiral purity.

In one embodiment, the present disclosure provides a method of preparing a chiral tryptophan compound or a pharmaceutically acceptable salt, polymorph, hydrate, solvate, tautomer, or stereoisomer thereof of Formula IVb:

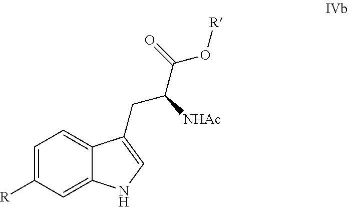

wherein R is halogen; and

wherein R′ is selected from the group consisting of alkyl and substituted alkyl;

the method comprising:

a) enantioselectively hydrogenating an unsaturated tryptophan compound of Formula IIIb with a chiral catalyst to afford the chiral tryptophan compound of Formula IVb:

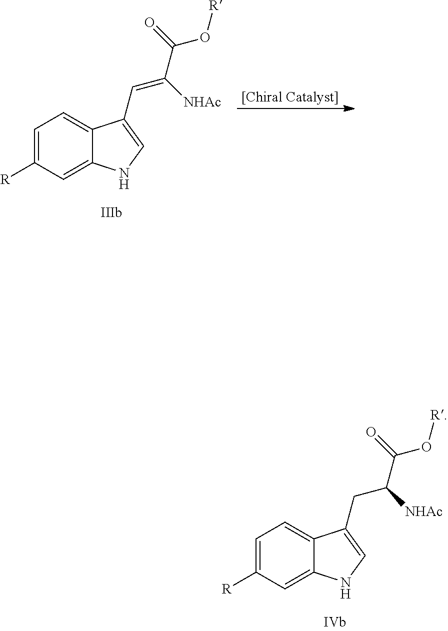

In some embodiments, R′ is an alkyl selected from the group consisting of methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. In other embodiments, R is a halogen selected from the group consisting of fluoro, chloro, bromo, and iodo. In other embodiments, the chiral catalyst comprises a chiral rhodium catalyst. In other embodiments, the chiral rhodium catalyst comprises a chiral phosphine ligand and rhodium. In other embodiments, the chiral phosphine ligand is an enantiomer of DuanPhos or an enantiomer of DuPhos. In other embodiments, the chiral phosphine ligand is an enantiomer of DuanPhos. In other embodiments, the chiral tryptophan compound of Formula IVb is (S)-ethyl 2-acetamido-3-(6-chloro-1H-indol-3-yl)propanoate.

In another embodiment, the present disclosure provides a method of preparing a compound of Formula I or a pharmaceutically acceptable salt, polymorph, hydrate, solvate, tautomer, or stereoisomer thereof:

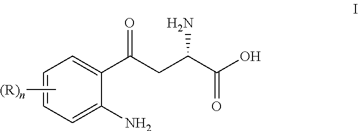

wherein each R is independently selected from the group consisting of hydrogen, halogen, alkyl, and substituted alkyl; and

wherein n=0-4;

the method comprising:

a) coupling an indole aldehyde compound of Formula II with an acetamidomalonate compound of Formula IIa in the presence of a suitable anhydride compound in a suitable solvent to afford an unsaturated tryptophan compound of Formula IIIa:

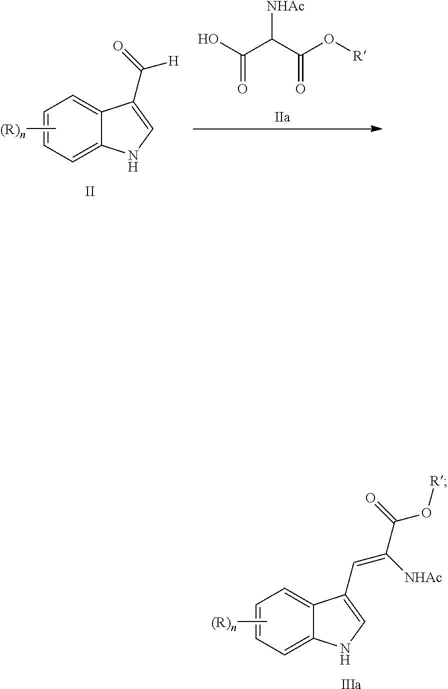

wherein R′ is selected from the group consisting of alkyl and substituted alkyl;

b) enantioselectively hydrogenating the unsaturated tryptophan compound of Formula IIIa with a chiral catalyst to afford the chiral tryptophan compound of Formula IVa:

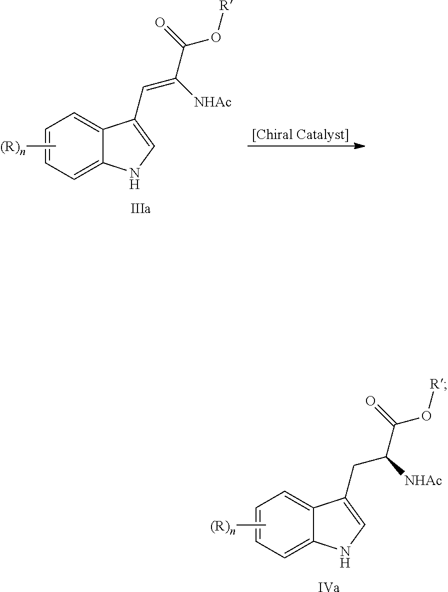

c) oxidizing the chiral tryptophan compound of Formula IVa with an oxidizing agent to afford a compound of Formula V:

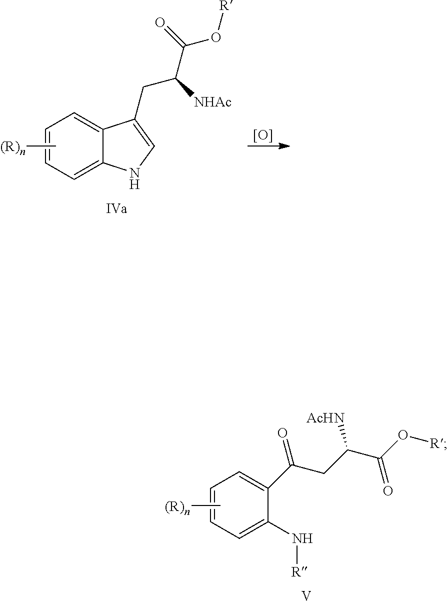

wherein R″ is selected from the group consisting of formyl and hydrogen; and

d) deprotecting the compound of Formula V to afford the compound of Formula I:

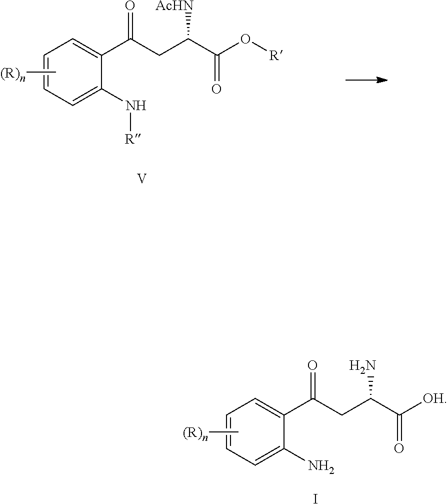

In some embodiments, R′ is an alkyl selected from the group consisting of methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. In other embodiments, n=1 and R is a halogen selected from the group consisting of fluoro, chloro, bromo, and iodo. In other embodiments, in step a) the suitable anhydride compound is acetic anhydride and the suitable solvent is pyridine. In other embodiments, the chiral catalyst comprises a chiral rhodium catalyst. In other embodiments, the chiral rhodium catalyst comprises a chiral phosphine ligand and rhodium. In other embodiments, the chiral phosphine ligand is an enantiomer of DuPhos or DuanPhos. In other embodiments, the chiral phosphine ligand is an enantiomer of DuanPhos. In other embodiments, the oxidizing agent is selected from the group consisting of m-chloroperoxybenzoic acid, potassium peroxysulfate, sodium periodate, ozone, superoxide, peracetic acid, and RuCl₃/sodium periodate. In other embodiments, the oxidizing agent is m-chloroperoxybenzoic acid. In other embodiments, the deprotecting comprises heating the compound of Formula V in the presence of HCl. In other embodiments, the deprotecting comprises heating the compound of Formula V in the presence of HCl followed by addition of sulfuric acid and isolation of the compound of Formula I as a sulfate monohydrate salt. In other embodiments, the sulfate monohydrate salt is reacted with sodium hydroxide to afford the compound of Formula I as a free base. In other embodiments, the sulfate monohydrate salt is reacted with Amberlite resin to afford the compound of Formula I as a free base. In other embodiments, the compound of Formula I is L-4-chlorokynurenine.

In another embodiment, the present disclosure provides a method of preparing the compound:

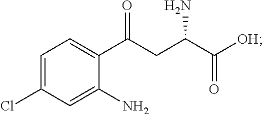 [SMILES: Nc1cc(Cl)ccc1C(=O)C[C@H](N)C(=O)O]

the method comprising:

a) coupling 6-chloroindole-3-carboxaldehyde with ethyl acetamidomalonate in the presence of acetic anhydride in pyridine solvent to afford ethyl Z-α-acetamido-6-chloroindole-3-acrylate:

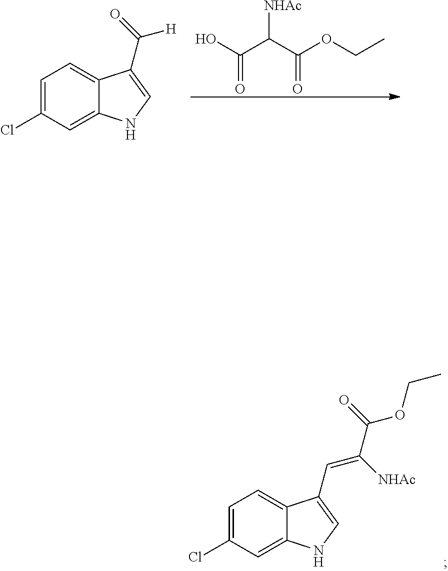

b) enantioselectively hydrogenating ethyl Z-α-acetamido-6-chloroindole-3-acrylate with [(S,S′,R,R′-DuanPhos)Rh(COD)][BF₄] to afford (S)-ethyl 2-acetamido-3-(6-chloro-1H-indol-3-yl)propanoate:

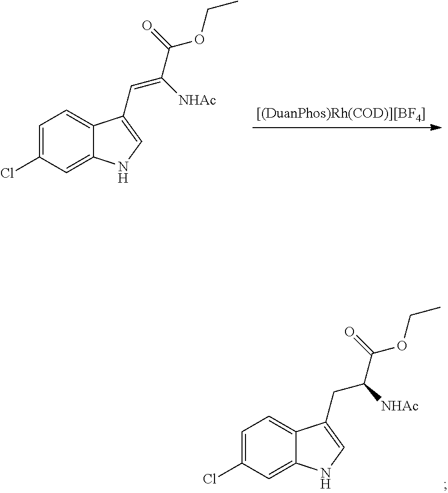 [SMILES: CCOC(=O)/C(=C/c1c[nH]c2cc(Cl)ccc12)NC(C)=O ; CCOC(=O)[C@H](Cc1c[nH]c2cc(Cl)ccc12)NC(C)=O]

c) oxidizing (S)-ethyl 2-acetamido-3-(6-chloro-1H-indol-3-yl)propanoate with m-chloroperoxybenzoic acid (MCPBA) to afford ethyl (2S)-2(acetamido)-4-(2-carbonylamino-4-chlorophenyl)-4-oxobutanoate:

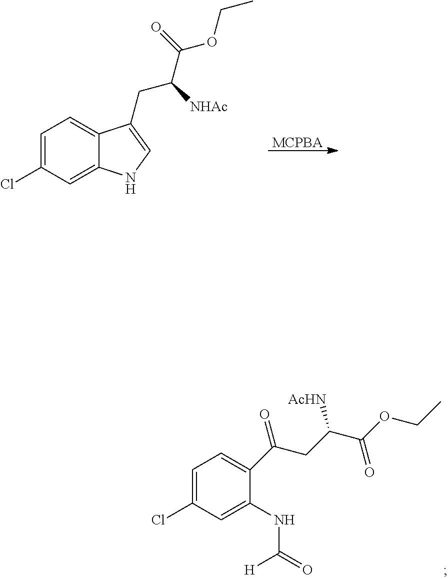

and

d) deprotecting ethyl (2S)-2(acetamido)-4-(2-carbonylamino-4-chlorophenyl)-4-oxobutanoate to afford L-4-chlorokynurenine:

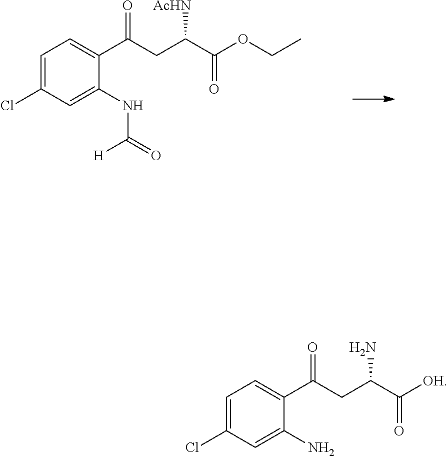 [SMILES: CCOC(=O)[C@H](CC(=O)c1ccc(Cl)cc1NC=O)NC(C)=O ; Nc1cc(Cl)ccc1C(=O)C[C@H](N)C(=O)O]

In some embodiments, the deprotecting step comprises generating L-4-chlorokynurenine sulfate monohydrate. In other embodiments, the sulfate salt of the L-4-chlorokynurenine sulfate monohydrate is removed to afford L-4-chlorokynurenine.

The present disclosure also provides a compound having the structure:

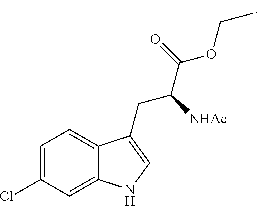 [SMILES: CCOC(=O)[C@H](Cc1c[nH]c2cc(Cl)ccc12)NC(C)=O]

The present disclosure also provides a compound having the structure:

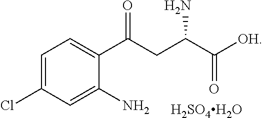

In some embodiments, the present disclosure also provides a pharmaceutical composition comprising the compound of Formula I, L-4-chlorokynurenine sulfate monohydrate, L-4-chlorokynurenine, or any pharmaceutically acceptable salt, polymorph, hydrate, solvate, tautomer, or stereoisomer thereof. In some embodiments, the compound of Formula I, L-4-chlorokynurenine sulfate monohydrate, L-4-chlorokynurenine, or any pharmaceutically acceptable salt, polymorph, hydrate, solvate, tautomer, or stereoisomer thereof has at least about 95% chemical purity and at least about 95% ee. In some embodiments, the compound of Formula I, L-4-chlorokynurenine sulfate monohydrate, L-4-chlorokynurenine, or any pharmaceutically acceptable salt, polymorph, hydrate, solvate, tautomer, or stereoisomer thereof is prepared by the methods disclosed herein.

### DETAILED DESCRIPTION

Provided are methods of preparing compounds, including chiral kynurenine compounds, as well as intermediates useful for the synthesis of the compounds.

### DEFINITIONS

The term “alkyl” includes saturated aliphatic groups including straight-chain, branched-chain, cyclic groups, and combinations thereof. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cycloalkyl groups can consist of one ring, including, but not limited to, groups such as cycloheptyl, or multiple fused rings, including, but not limited to, groups such as adamantyl or norbornyl.

“Substituted alkyl” includes alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂CF₃, and other perfluoro and perhalo groups; —CH₂—OH; —CH₂CH₂CH(NH₂)CH₃, etc.

The term “halogen” as used herein includes the Group VIIa elements (Group 17 elements in the 1990 International Union of Pure and Applied Chemistry (IUPAC) Periodic Table, IUPAC Nomenclature of Inorganic Chemistry, Recommendations 1990) and includes fluoro, chloro, bromo, and iodo substituents.

The term “polymorph” refers to a compound that occurs in two or more forms, such as, for example, two or more crystalline forms.

The term “chemical purity” refers to the overall level of a desired product or compound in a composition produced by a preparation. If a compound is present in enantiomeric forms, “chemical purity” as used herein would include both enantiomeric forms in the calculation of the overall level of the desired product. The components of the composition other than the desired product or compound are “impurities.” The purity may be measured a variety of techniques, including HPLC analysis.

The term “enantiomeric purity” or “chiral purity” refers to the overall level of one enantiomer in a composition as compared to the other enantiomer in the composition. Components of the composition other than either of the enantiomers are not considered in the calculation of “enantiomeric purity” or “chiral purity.” The enantiomeric purity or chiral purity may be measured by a variety of techniques, including chiral HPLC analysis.

The term “cc” refers to “enantiomeric excess” as calculated by the following: (moles of one enantiomer−moles of other enantiomer)/moles of both enantiomers×100.

A “therapeutically effective amount” of a compound is an amount of the compound, which when administered to a subject, is sufficient to prevent, reduce, eliminate, retard the progression of, or reduce the severity of the disease, disorder, or condition or one or more symptoms of the disease, disorder, or condition.

### Compounds

A variety of compounds, including chiral kynurenine compounds, may be synthesized using the methods disclosed herein. In some embodiments, a compound of Formula I may be synthesized, or a pharmaceutically acceptable salt, polymorph, hydrate, solvate, tautomer, or stereoisomer thereof:

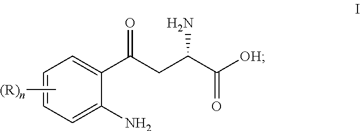

wherein each R is independently selected from the group consisting of hydrogen, halogen, alkyl, and substituted alkyl; and

wherein n=0 to 4, or preferably 1 or 2.

Where a chiral center is shown, any stereoisomer is within the scope of the invention. Where an (S) isomer is disclosed, the corresponding (R) isomer is within the scope of the invention. Where an (L) isomer is disclosed, the corresponding (D) isomer is within the scope of the invention. Where an (R) isomer is disclosed, the corresponding (S) isomer is within the scope of the invention. Where a (D) isomer is disclosed, the corresponding (L) isomer is with in the scope of the invention.

In a particular embodiment the compound is L-4-chlorokynurenine, which also is referred to by the chemical name (S)-2-amino-4-(2-amino-4-chlorophenyl)-4-oxobutanoic acid:

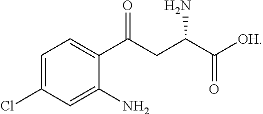 [SMILES: Nc1cc(Cl)ccc1C(=O)C[C@H](N)C(=O)O]

In another embodiment, a compound that is useful as an intermediate for the synthesis of the compound of Formula I, L-4-chlorokynurenine sulfate monohydrate, L-4-chlorokynurenine, or any pharmaceutically acceptable salt, polymorph, hydrate, solvate, tautomer, or stereoisomer thereof is provided, which may be synthesized as disclosed herein. In one embodiment, the compound is of Formula IVa:

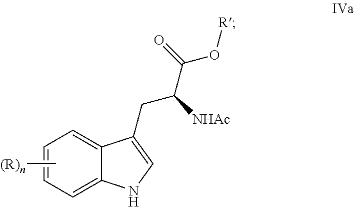

wherein each R is independently selected from the group consisting of hydrogen, halogen, alkyl, and substituted alkyl;

wherein R′ is selected from the group consisting of alkyl and substituted alkyl; and

wherein n=0 to 4, or preferably 1 or 2.

In another embodiment, the compound useful as an intermediate is of Formula IVb:

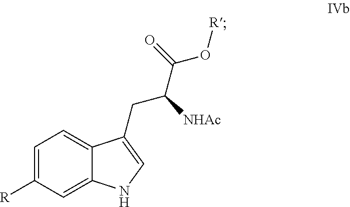

wherein R is halogen; and

wherein R′ is selected from the group consisting of alkyl and substituted alkyl.

In a particular embodiment, the compound useful as an intermediate is (S)-ethyl 2-acetamido-3-(6-chloro-1H-indol-3-yl)propanoate:

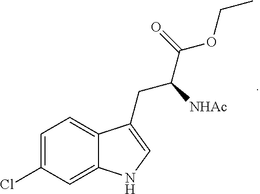

The compounds described herein may optionally be in the form of a salt, such as a pharmaceutically acceptable salt. The pharmaceutically acceptable salt may in certain embodiments optionally impart improved pharmacokinetic properties on the active ingredient compared with the free form of the compound. The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, lithium salts and calcium salts; ammonium salts; and aluminum salts. Sulfate salts are also contemplated. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared.

Where a compound is described herein, all stereoisomers thereof are also contemplated, including diastereomers and enantiomers, as well as mixtures of stereoisomers, including, but not limited to, racemic mixtures.

Some compounds of the present invention may exhibit polymorphism. The scope of the present invention includes all polymorphic forms of the compounds according to the invention.

### Methods of Preparation and Intermediate Compounds

Methods of preparing a compound, for example of Formula I, or a pharmaceutically acceptable salt, polymorph, hydrate, solvate, tautomer, or stereoisomer thereof are provided:

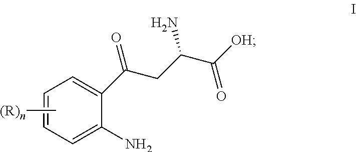

wherein each R is independently selected from the group consisting of hydrogen, halogen, alkyl, and substituted alkyl; and

wherein n=0 to 4, or preferably 1 or 2.

In one embodiment, a method of preparing a chiral tryptophan intermediate compound of Formula IVa or a pharmaceutically acceptable salt, polymorph, hydrate, solvate, tautomer, or stereoisomer thereof is provided:

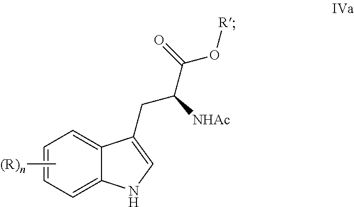

wherein each R is independently selected from the group consisting of hydrogen, halogen, alkyl, and substituted alkyl;

wherein R′ is selected from the group consisting of alkyl and substituted alkyl; and

wherein n=0-4, or preferably 1 or 2.

In another embodiment, a method of preparing a chiral tryptophan intermediate compound of Formula IVb or a pharmaceutically acceptable salt, polymorph, hydrate, solvate, tautomer, or stereoisomer thereof is provided:

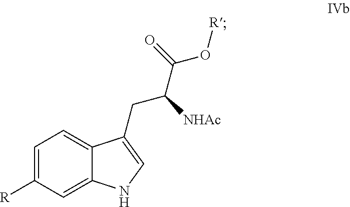

wherein R is halogen; and

wherein R′ is selected from the group consisting of alkyl and substituted alkyl.

The compounds of Formula IVa and IVb are useful in some embodiments as intermediates in the preparation of the compound of Formula I, L-4-chlorokynurenine sulfate monohydrate, L-4-chlorokynurenine, or any pharmaceutically acceptable salt, polymorph, hydrate, solvate, tautomer, or stereoisomer thereof.

In a particular embodiment, a compound of Formula I is prepared by transformation of a chiral tryptophan intermediate compound or a pharmaceutically acceptable salt, polymorph, hydrate, solvate, tautomer, or stereoisomer thereof of Formula IVa or IVb.

In a particular embodiment, a compound of Formula IVa may be prepared by enantioselectively hydrogenating an unsaturated tryptophan compound of Formula IIIa with a chiral catalyst to afford the chiral tryptophan compound of Formula IVa:

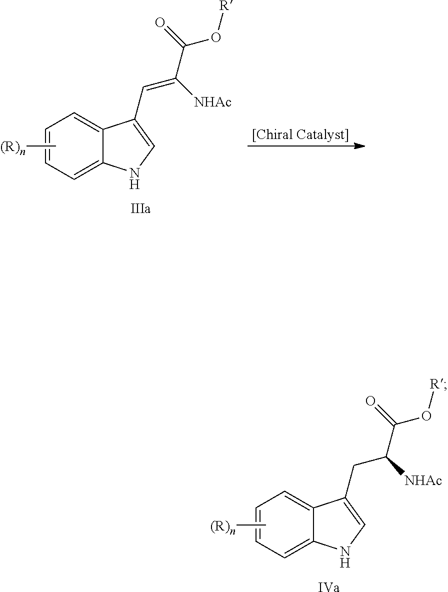

wherein each R is independently selected from the group consisting of hydrogen, halogen, alkyl, and substituted alkyl;

wherein R′ is selected from the group consisting of alkyl and substituted alkyl; and

wherein n=0 to 4, or preferably 1 or 2.

In another particular embodiment, a compound of Formula IVb may be prepared by enantioselectively hydrogenating an unsaturated tryptophan compound of Formula IIIb with a chiral catalyst to afford the chiral tryptophan compound of Formula IVb:

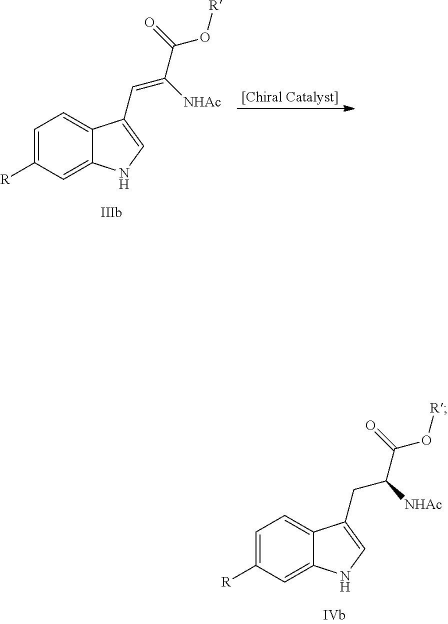

wherein R is halogen; and

wherein R′ is selected from the group consisting of alkyl and substituted alkyl.

In another particular embodiment, R′ is an alkyl selected from the group consisting of methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. In another particular embodiment R is a halogen selected from the group consisting of fluoro, chloro, bromo, and iodo.

Also provided is a method of preparing a compound of Formula I, such as a chiral kynurenine compound, or a pharmaceutically acceptable salt, polymorph, hydrate, solvate, tautomer, or stereoisomer thereof:

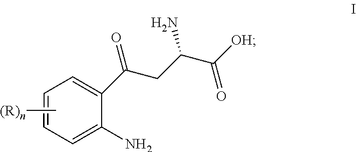

wherein each R is independently selected from the group consisting of hydrogen, halogen, alkyl, and substituted alkyl; and

wherein n=0-4;

the method comprising:

a) coupling an indole aldehyde compound of Formula II with an acetamidomalonate compound of Formula IIa in the presence of a suitable anhydride compound in a suitable solvent to afford an unsaturated tryptophan compound of Formula IIIa:

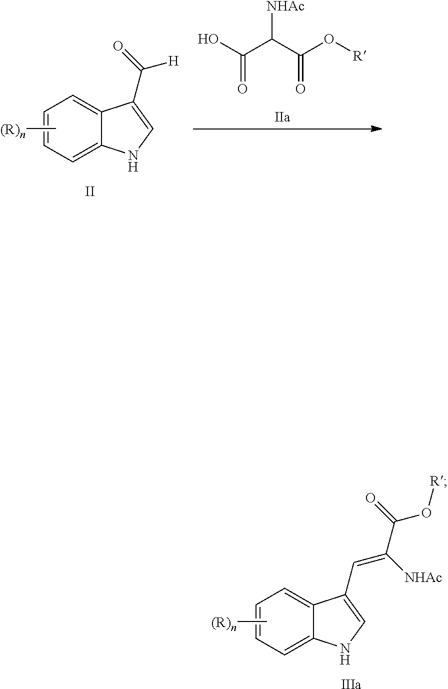

wherein R′ is selected from the group consisting of alkyl and substituted alkyl;

b) enantioselectively hydrogenating the unsaturated tryptophan compound of Formula IIIa with a chiral catalyst to produce the chiral tryptophan compound of Formula IVa:

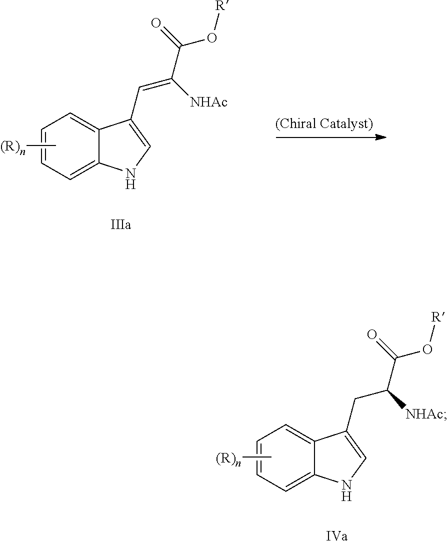

c) oxidizing the chiral tryptophan compound of Formula IVa with an oxidizing agent to afford a compound of Formula V:

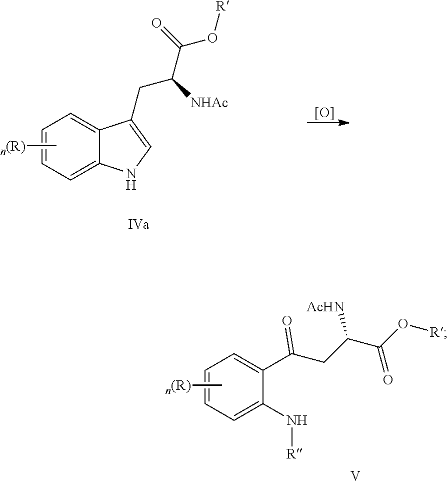

wherein R″ is selected from the group consisting of formyl and hydrogen; and

d) deprotecting the compound of Formula V to afford the compound of Formula I:

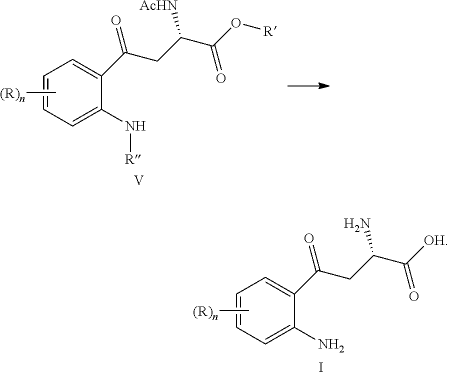

The R enantiomers of the compounds of Formulas I, IVa, IVb, and V are also contemplated.

In one embodiment, R′ is an alkyl selected from the group consisting of methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl.

In another embodiment, n=1 and R is a halogen selected from the group consisting of fluoro, chloro, bromo, and iodo.

In another embodiment, R″ is formyl.

In one embodiment of step a), the suitable anhydride compound is acetic anhydride and the suitable solvent is pyridine.

In some embodiments, the chiral catalyst is a chiral, transition metal hydrogenation catalyst. In one embodiment of step b), the chiral catalyst is a rhodium catalyst formed from the reaction of a phosphine ligand and a rhodium precursor. The phosphine ligand may be an enantiomer of DuanPhos [DuanPhos=(−)-(1S,1′S,2R,2′R)-2,2′-di-tert-butyl-2,3,2′,3′-tetrahydro-1H,1′H-(1,1′)biisophosphindolyl or (+)-(1R,1′R,2S,2′S)-2,2′-di-tert-butyl-2,3,2′,3′-tetrahydro-1H,1′H-(1,1′)biisophosphindolyl] or an enantiomer of DuPhos [DuPhos=(−)-1,2-bis((2R,5R)-2,5-diethylphospholano)benzene or (+)-1,2-bis((2S,5S)-2,5-diethylphospholano)benzene].

In another embodiment, the rhodium precursor may be [Rh(NBD)₂]X; [Rh(NBD)Cl]]₂; [Rh(COD)Cl]₂; [Rh(COD)₂]X; [Rh(acac)(CO)₂]; [Rh(ethylene)₂(acac)]; [Rh(ethylene)₂Cl]₂; [RhCl(PPh₃)₃]; or [Rh(CO)₂Cl₂] wherein acac=acetylacetonate, NBD=norbornadiene, COD=cyclooctadiene, and the counteranion X may be halogen, BF₄, ClO₄, SbF₆, PF₆, CF₃SO₃, RCOO, or B(Ar)₄, wherein Ar is fluorophenyl or 3,5-di-trifluoromethyl-1-phenyl. Mixtures of counteranion X are also contemplated.

In other embodiments, commercially available, preformed catalysts such as [(DuanPhos)Rh(COD)][BF₄] and [(DuPhos)Rh(COD)][BF₄] may also be used. The DuanPhos ligand is disclosed in U.S. Pat. Nos. 7,153,809 and 7,169,953.

In some embodiments, the reaction with the chiral catalyst may result in acylated tryptophan and acylated indole as byproducts. In some embodiments, manufacturing steps to recover and recycle the acylated byproducts may be used to increase the overall yield of the desired compounds.

In one embodiment of step c) the oxidizing agent is m-chloroperoxybenzoic acid (MCPBA). In another embodiment of step c), the oxidizing agent is potassium peroxysulfate, sodium periodate, ozone, superoxide, peracetic acid, or RuCl₃/sodium periodate.

In one embodiment of step d), the deprotecting includes heating the compound of Formula V in the presence of HCl followed by work-up with an excess amount of sodium hydroxide to form the sodium salt form. The excess base then can require an equivalent amount of hydrochloric acid to precipitate the free base product. An alternative deprotection route may involve heating the compound of Formula V in the presence of HCl followed by addition of sulfuric acid and isolation of the compound of Formula VI (the sulfate monohydrate salt of the compound of Formula I) with high chiral and chemical purities. This method can eliminate the excess sodium hydroxide and hydrochloric acid and generation of inorganic salts. In some embodiments, the salt form may be easily isolated on a filter funnel. In some embodiments, the sulfate salt of the compound of Formula VI may be removed to produce the compound of Formula I by reacting with caustic soda and precipitating with an organic solvent such as acetonitrile or acetone as an anti-solvent. In one preferred embodiment, the sulfate salt of the compound of Formula VI may be removed to produce the compound of Formula I by treatment with a resin, such as, for example, an Amberlite resin (FPA 53 resin) as shown in the following exemplary reaction scheme:

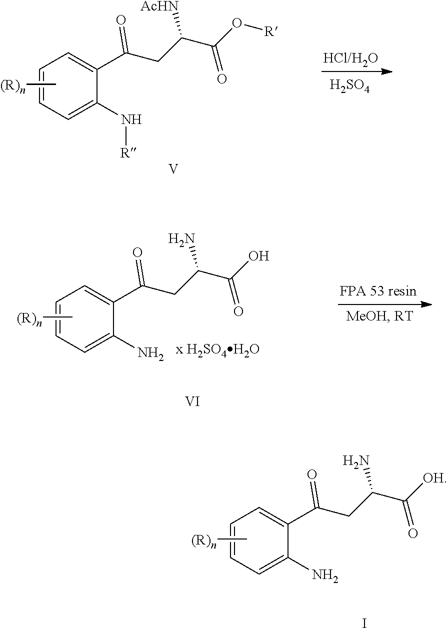

In a particular embodiment, the compound of Formula IVa or IVb useful as an intermediate in the preparation of the compound L-4-chlorokynurenine may be (S)-ethyl 2-acetamido-3-(6-chloro-1H-indol-3-yl)propanoate. In some embodiments, (S)-ethyl 2-acetamido-3-(6-chloro-1H-indol-3-yl)propanoate may be prepared by enantioselectively hydrogenating ethyl Z-α-acetamido-6-chloroindole-3-acrylate with [(S,S′,R,R′-DuanPhos)Rh(COD)][BF₄] to afford (S)-ethyl 2-acetamido-3-(6-chloro-1H-indol-3-yl)propanoate:

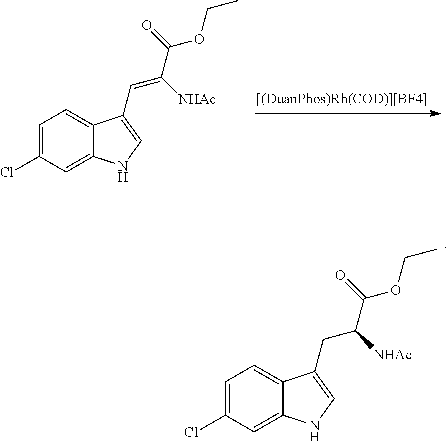 [SMILES: CCOC(=O)/C(=C/c1c[nH]c2cc(Cl)ccc12)NC(C)=O ; CCOC(=O)[C@H](Cc1c[nH]c2cc(Cl)ccc12)NC(C)=O]

In another embodiment, provided is a method of preparing the chiral kynurenine compound L-4-chlorokynurenine:

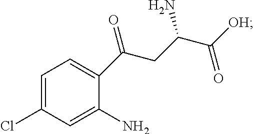 [SMILES: Nc1cc(Cl)ccc1C(=O)C[C@H](N)C(=O)O]

wherein the method comprises:

a) coupling 6-chloroindole-3-carboxaldehyde with ethyl acetamidomalonate in the presence of acetic anhydride in pyridine solvent to afford ethyl Z-α-acetamido-6-chloroindole-3-acrylate:

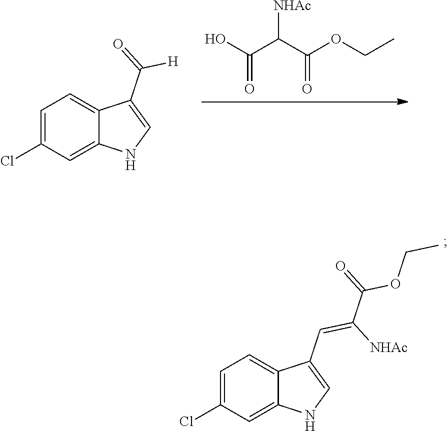 [SMILES: O=Cc1c[nH]c2cc(Cl)ccc12 ; CCOC(=O)C(NC(C)=O)C(=O)O ; CCOC(=O)/C(=C/c1c[nH]c2cc(Cl)ccc12)NC(C)=O]

b) enantioselectively hydrogenating ethyl Z-α-acetamido-6-chloroindole-3-acrylate with [(S,S′,R,R′-DuanPhos)Rh(COD)][BF₄] to afford (S)-ethyl 2-acetamido-3-(6-chloro-1H-indol-3-yl)propanoate:

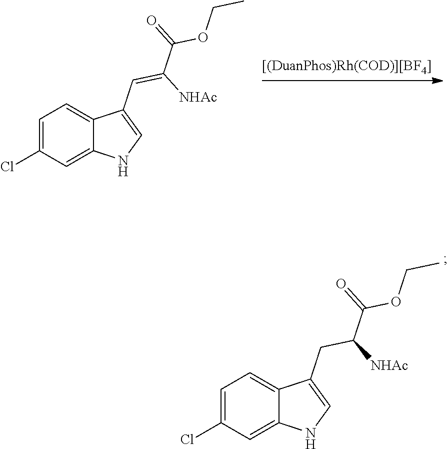 [SMILES: CCOC(=O)/C(=C/c1c[nH]c2cc(Cl)ccc12)NC(C)=O ; CCOC(=O)[C@H](Cc1c[nH]c2cc(Cl)ccc12)NC(C)=O]

c) oxidizing (S)-ethyl 2-acetamido-3-(6-chloro-1H-indol-3-yl)propanoate with MCPBA to afford ethyl (2S)-2(acetamido)-4-(2-carbonylamino-4-chlorophenyl)-4-oxobutanoate:

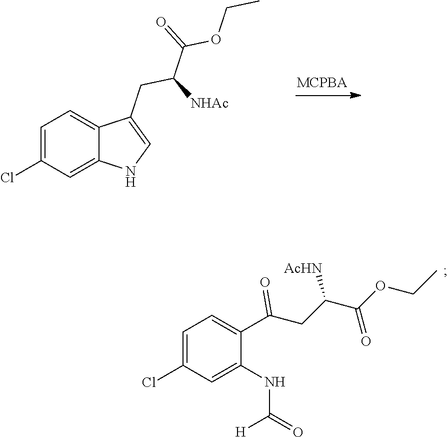 [SMILES: CCOC(=O)[C@H](Cc1c[nH]c2cc(Cl)ccc12)NC(C)=O ; CCOC(=O)[C@H](CC(=O)c1ccc(Cl)cc1NC=O)NC(C)=O]

and

d) deprotecting ethyl (2S)-2(acetamido)-4-(2-carbonylamino-4-chlorophenyl)-4-oxobutanoate to afford L-4-chlorokynurenine:

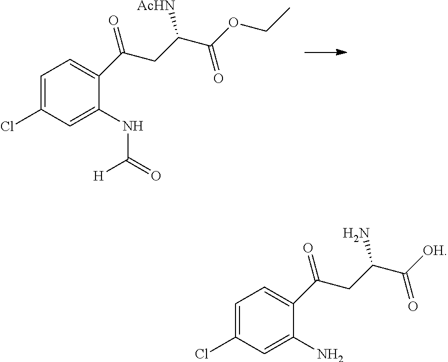 [SMILES: CCOC(=O)[C@H](CC(=O)c1ccc(Cl)cc1NC=O)NC(C)=O ; Nc1cc(Cl)ccc1C(=O)C[C@H](N)C(=O)O]

In certain embodiments, the methods allow for the production of compositions comprising compounds in high chemical purity, high enantiomeric purity, or in high enantiomeric excess. In some embodiments, a composition comprising the compound of Formula I, L-4-chlorokynurenine sulfate monohydrate, L-4-chlorokynurenine, or any pharmaceutically acceptable salt, polymorph, hydrate, solvate, tautomer, or stereoisomer thereof is provided in a range of about 95% to about 100% for both chemical purity and enantiomeric excess. In some embodiments, a composition comprising the compound of Formula I, L-4-chlorokynurenine sulfate monohydrate, L-4-chlorokynurenine, or any pharmaceutically acceptable salt, polymorph, hydrate, solvate, tautomer, or stereoisomer thereof is provided in about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% chemical purity and/or in about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% enantiomeric excess (ee).

In certain embodiments, compositions are provided comprising L-4-chlorokynurenine in a range of about 95% to about 100% for both chemical purity and enantiomeric excess. In certain embodiments, compositions are provided comprising L-4-chlorokynurenine in about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% chemical purity and/or in about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% ee.

In certain embodiments, compositions are provided comprising the sulfate salt of L-4-chlorokynurenine in a range of about 95% to about 100% for both chemical purity and enantiomeric excess. In certain embodiments, compositions are provided comprising the sulfate salt of L-4-chlorokynurenine in about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% chemical purity and/or in about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% ee.

### Methods of Use

The compounds disclosed herein can be used in a variety of therapeutic applications. One such use is in the treatment of neuropathic pain.

L-4-chlorokynurenine and other chiral kynurenine compounds offer a valuable treatment option to patients with neuropathic pain, including patients with neuropathic pain complications from infection with HIV.

Thus in certain embodiments, methods of treatment of neuropathic pain are provided comprising administering a therapeutically effective amount of the compound of Formula I, L-4-chlorokynurenine sulfate monohydrate, L-4-chlorokynurenine, or any pharmaceutically acceptable salt, polymorph, hydrate, solvate, tautomer, or stereoisomer thereof or a pharmaceutical composition comprising the compound of Formula I, L-4-chlorokynurenine sulfate monohydrate, L-4-chlorokynurenine, or any pharmaceutically acceptable salt, polymorph, hydrate, solvate, tautomer, or stereoisomer thereof. The subjects which can be treated include vertebrates, preferably mammals, and more preferably humans. The compounds disclosed herein and pharmaceutical compositions comprising the compounds may be used in manufacture of a medicament for treatment of neuropathic pain.

The compounds described herein can be administered to a mammal, preferably human, subject via any route known in the art, including, but not limited to, those disclosed herein. Methods of administration include but are not limited to, intravenous, oral, intra-arterial, intramuscular, topical, via inhalation (e.g. as mists or sprays), via nasal mucosa, subcutaneous, transdermal, intraperitoneal, gastrointestinal, and rectal. Oral administration is a preferred route of administration.

Pharmaceutical compositions comprising the compounds disclosed herein, including the compound of Formula I, L-4-chlorokynurenine sulfate monohydrate, L-4-chlorokynurenine, or any pharmaceutically acceptable salt, polymorph, hydrate, solvate, tautomer, or stereoisomer thereof, are also provided, which optionally may include the compound in combination with a pharmaceutically acceptable carrier, such as an organic carrier or inorganic carrier. The pharmaceutical unit dosage chosen is preferably fabricated and administered to provide a defined final concentration of drug in the blood, tissues, organs, or other targeted region of the body. The optimal effective concentration of the compounds of the invention can be determined empirically and will depend on the type and severity of the disease, disorder, or condition; route of administration; disease, disorder, or condition progression and health; and mass and body area of the patient. Such determinations are within the skill of one in the art.

The invention will be further understood by the following non-limiting examples.

### Example 1

### Preparation of Ethyl Z-α-acetamido-6-chloroindole-3-acrylate

6-chloroindole-3-carboxaldehyde (530 g, 2.95 mol), ethyl acetamidomalonate (837 g, 4.42 mol, 1.50 equiv), and pyridine (2650 mL) were charged to a 5 L 3-neck round bottom flask, agitated, and heated to 25° C. until a clear solution was formed. The resulting solution was cooled to −5° C. and acetic anhydride (964 g, 9.44 mol, 3.20 equiv) was slowly added over 3-4 hours at <0° C. The resulting reaction mixture was warmed to room temperature and stirred overnight. The reaction progress was monitored by partitioning a sample of the reaction mixture between dilute aqueous hydrochloric acid (HCl) and tetrahydrofuran (THF). The organic layer was spotted on two TLC plates and run in 100% ethyl acetate and 50/50 ethyl acetate/heptane mobile phases. In 100% ethyl acetate; product Rf 0.26, acylated product Rf 0.35. In 50/50 ethyl acetate/heptane; starting material Rf 0.23, acylated starting material Rf 0.30, and product Rf 0.04. The starting material was typically fully converted after stirring for approximately 20 hours. The resulting slurry was again cooled to −5° C. and filtered. The solids were washed with cold pyridine two times to afford an off-white N-acylated indole aldehyde. The dry weight of this recovered N-acylated starting material was 256 g (1.15 mol). The filtrate was added to water (26.5 L, 10 times the volume of pyridine) with stirring. The filtrate was added at such a rate to maintain the temperature <25° C. Sodium carbonate (1251 g) was added portion-wise to control foaming and to maintain the temperature <25° C. The slurry was cooled to 5° C. after the addition of sodium carbonate was complete. The crude solids were filtered, washed with water, and pulled dry (Wet weight 718 g). The crude solids were then slurried in water (14.4 L, 20 volumes wet weight) and acetonitrile (3.6 L, 5 volumes wet weight). Caustic soda (80 mL) was added to the slurry to adjust the pH to >12. TLC monitoring (100% ethyl acetate) illustrated that after stirring for 4 hours, the N-acylated product had been fully converted to the desired product. Concentrated HCl (130 mL) was added to the slurry to adjust the pH to 5-6. The product was then filtered, washed with water, and then washed with methyl tert-butyl ether (MTBE) three times. 10.6 g of product was dried at 45° C. and at <10 mm Hg in a vacuum oven. Yield of ethyl Z-α-acetamido-6-chloroindole-3-acrylate: 407 g (1.33 mol, 45% yield, 74% yield corrected for recovered starting material). HPLC: 97.4% chemical purity. Yield of recovered N-acylated indole aldehyde: 256 g (1.15 mol, equates to 207 g of indole aldehyde). HPLC: 98.5% chemical purity.

In an alternative version of the above reaction, the sodium carbonate workup was replaced with an acidic workup. The acidic workup resulted in easier processing and reduced cycle time.

### Example 2

### Preparation of (S)-ethyl 2-acetamido-3-(6-chloro-1H-indol-3-yl)propanoate

Ethyl Z-α-acetamido-6-chloroindole-3-acrylate (434 g, 1.41 mol) was dissolved in methanol, AR (AR=anhydrous reagent grade, 6510 mL) at 60° C. to form a light yellow solution. This solution was then cooled to room temperature and charged to a 20 L hydrogenator. The hydrogenator was purged with 25 psi of nitrogen three times and allowed to stir under 25 psi of nitrogen for 20 minutes to remove any dissolved oxygen from the reaction mixture. The hydrogenator was then vented and the chiral catalyst [(S,S′,R,R′)-DuanPhosRh(COD)][BF₄] (0.93 g, 0.0014 mol, 0.001 equiv) was charged to the reaction mixture. The hydrogenator was pressurized with 30 psi of hydrogen gas and stirred for 10 minutes. The gas was then vented and the procedure was repeated two more times. The hydrogenator was re-pressurized with 90 psi of hydrogen gas then stirred at room temperature overnight. The hydrogenator was periodically re-pressurized to 90 psi as hydrogen was consumed. The reaction progress was monitored by directly spotting the mixture on a TLC plate. In 100% ethyl acetate; product Rf 0.33, starting material Rf 0.26. The total time typically required for the starting material to fully convert to product was 20 to 24 hours. The reaction mixture was removed from the hydrogenator and concentrated on a Buchi rotoevaporator to dryness at a bath temperature of 35° C. to 40° C. The crude residue was then dissolved in methanol, AR (1302 mL, 3 volumes of starting material) at −60° C. Activated carbon (˜5 wt % of starting material) was added to the hot solution, and the resulting reaction mixture was stirred to a uniform suspension and filtered through a double layer glass fiber filter. The filtrate was cooled to <0° C. to precipitate the product. This slurry was then placed in the freezer overnight (−20° C.) to further precipitate the product. The slurry was vigorously stirred and filtered. The solids were then removed from the filter and re-slurried in MTBE. The slurry was again filtered, and the solids were washed with MTBE and pulled dry on the filter. The product was placed on drying trays and dried in a vacuum oven at 35° C.<10 mm Hg) until all solvent was removed. Yield of 1^{st} crop: 326 g (1.06 mol, 75%, off-white solid). HPLC: 99.5% chemical purity, 100% ee. The filtrate and washes from the first crop were concentrated to dryness to yield 111 g of crude solids. These solids were dissolved in methanol, AR (333 mL, 3 volumes crude solids) at 60° C. The hot solution was treated with activated carbon (˜5 wt % crude solids) and then filtered through a double layer glass fiber filter. The filtrate was cooled to <0° C. to precipitate the product. This slurry was then placed in the freezer overnight (−20° C.) to further precipitate the product. The slurry was vigorously stirred and filtered. The solids were then removed from the filter and re-slurried in MTBE. The slurry was again filtered, washed with MTBE, and pulled dry on the filter. The product was placed on a drying tray and dried in a vacuum oven at 35° C. and at <10 mm Hg until all solvent was removed. Yield of 2^{nd} crop: 75 g (0.24 mol, 17%, off-white solid). HPLC: 96.9% chemical purity. Chiral HPLC: 99.9% ee.

In an alternative of the above reaction, the hydrogenator was conditioned by charging it with methanol and the catalyst and stirring overnight at room temperature and then discarding. Conditioning improved the overall yield of (S)-ethyl 2-acetamido-3-(6-chloro-1H-indol-3-yl)propanoate.

### Example 3

### Preparation of Ethyl (2S)-2-(acetamido)-4-(2-carbonylamino-4-chlorophenyl)-4-oxobutanoate

A 50 gallon reactor was charged with (S)-ethyl 2-acetamido-3-(6-chloro-1H-indol-3-yl)propanoate (3000 g, 9.716 mol) in THF (5 L) and dichloromethane (DCM, 63 L), heated to 30° C. to produce a clear solution, and then cooled to −20° C. and stirred at the maximum agitator speed. The solution was pH 10.0. A solution of MCPBA (4785 g, 27.73 mol, 2.854 equiv) in THF (2.5 L) and DCM (15 L) was prepared and charged to a 20 L addition funnel. Concurrently, a solution of sodium carbonate (2265 g) in water (12 L) was charged to a second 20 L addition funnel. The MCPBA solution was added at such a rate to maintain the temperature <−15° C. Once the reaction mixture reached pH 3-4, the sodium carbonate solution was added to maintain the reaction mixture between pH 3-6. Once all of the two solutions were added to the reactor, the reaction progress was checked by spotting the organic layer on a TLC plate. In 100% ethyl acetate; product Rf 0.29 and starting material Rf 0.33. Upon completion, as determined by TLC, a 15 wt % sodium carbonate solution (20 L) was added to the reaction mixture to quench the excess peroxide present. The reaction mixture was then warmed to 20° C. The layers were separated and the organic layer was collected in a PE crock. The aqueous layer was extracted with DCM (2×10 L). The combined organic layers were then washed with 15 wt % sodium carbonate solution (20 L) followed by brine (20 L). The organic layer was dried with sodium sulfate overnight. Charcoal (300 g, 10 wt % of starting material) was charged to the reaction mixture and stirred for 2 hours. The reaction mixture was filtered through a polypropylene filter pad and washed with DCM (2×5 L). The filtrate was concentrated in a reactor to the minimum stir volume at 20° C. under vacuum. The concentrate was then dropped out of the reactor and continued to concentrate on a Buchi rotoevaporator at 20-23° C. under high vacuum until solids formed. Isopropyl alcohol (IPA, 2 L) was added to the Buchi to co-evaporate the DCM from the solids. Concentration continued until all the DCM was removed from the crude solids. IPA (1 L) and MTBE (1 L) were added to the Buchi and the resulting slurry was cooled to 0° C. in an ice water bath. The solids were filtered, washed with cold 50/50 (IPA/MTBE) (3×1 L), washed with MTBE (2×1 L), and placed on drying trays in a vacuum oven at 28° C. and at <10 mm Hg overnight to remove all residual solvents. Yield: 1331 g (3.906 mol, 40%). HPLC: 98.9% chemical purity.

In optimization runs of the above reaction, prolonged addition rates of MCPBA and carbonate solution were demonstrated to have detrimental effects on chemical purity and yield. Faster addition of these reagents and tighter temperature controls improved the process. The IPA/MTBE slurry purification method was replaced with recrystallization from IPA alone, which reduced the number and level of impurities and provided a better HPLC profile of ethyl (2S)-2-(acetamido)-4-(2-carbonylamino-4-chlorophenyl)-4-oxobutanoate.

### Example 4

### Preparation of L-4-Chlorokynurenine

Ethyl (2S)-2-(acetamido)-4-(2-carbonylamino-4-chlorophenyl)-4-oxobutanoate (1891 g, 5.549 mol), water (10.4 L), and concentrated HCl (2774 mL, 33.29 mol, 6 equiv) were charged to a 22 L 3-neck round bottom flask in a heating mantle with a mechanical stirrer, thermocouple, nitrogen purge, and condenser. The mixture was heated to 70° C. and the resulting solution was held between 70-80° C. for 4 hours. After 4 hours, the heating mantle was turned off and activated carbon (189 g, 10 wt %) was added to the solution. The reaction mixture slowly cooled to room temperature while stirring overnight. The cooled reaction mixture was filtered through a glass fiber filter and basified to pH 12 with 50% sodium hydroxide. Solids precipitated in the pH 5˜6 range, but were redissolved as the reaction mixture became basic. The basic aqueous solution was washed with DCM (2×1.8 L), ethyl acetate (2×1.8 L), and MTBE (2×1.8 L). Concentrated HCl was added in portions to the aqueous reaction mixture to adjust to pH 6 and to maintain this pH. The resulting slurry was stirred out at room temperature overnight. The crude solids were collected by filtration on a centrifuge in batches and each batch was washed with acetonitrile (ACN, 2×500 mL). If multiple batches are being synthesized, as in the example described herein, the crude solids were not completely dried or analyzed before being combining and purifying as one, uniform lot.

All lots of the crude L-4-chlorokynurenine were combined and slurried in ACN (7 L) and water (1.4 L) overnight to reduce residual levels of sodium. The slurry was filtered on a centrifuge in batches. Each batch was spun dry and washed with ACN (3×500 mL). The solids were again slurried in ACN (7 L) and water (1.4 L) overnight. The slurry was filtered on the centrifuge in batches. Each batch was spun dry and washed with ACN (3×500 mL). For a third time, the solids were slurried in ACN (7 L) and water (1.4 L) for 1 hour. The slurry was filtered on the centrifuge in batches. Each batch was spun dry and washed with ACN (3×500 mL). The solids were slurried in a mixture of ethanol (200 proof) and ethyl acetate (50/50 mixture, 6 L) for 1 hour. The slurry was filtered on the centrifuge in batches. Each batch was spun dry and washed with a mixture of ethanol (200 proof) and ethyl acetate (50/50 mixture, 500 mL) followed by ethyl acetate (500 mL). The solids were placed on drying trays and dried in a vacuum oven at 30° C. (<10 mm Hg) overnight. The drying trays were removed from the oven and the solids were broken up by passing them through a sieve. Solids were placed back in the drying trays and returned to the vacuum oven to dry at 30° C. (<10 mm Hg) over the weekend. Yield: 2578 g (10.62 mol, 72%, off-white solid) from 5027 g (14.75 mol) of Ethyl (2S)-2-(acetamido)-4-(2-carbonylamino-4-chlorophenyl)-4-oxobutanoate. HPLC: 99.4% chemical purity. Chiral HPLC: 99.9% ee.

### Example 5

### Preparation of L-4-Chlorokynurenine Sulfate Monohydrate

Water, RO (RO=reverse osmosis, 100 mL), and concentrated HCl (29 mL, 0.35, 5.9 equiv) were charged to a 250 mL 3-neck round bottom flask in a heating mantle with a mechanical stirrer, thermocouple, nitrogen purge, and condenser. After the exotherm, ethyl (2S)-2-(acetamido)-4-(2-carbonylamino-4chlorophenyl)-4-oxobutanoate (20 g, 0.059 mol) was charged to the flask and an off-white slurry formed. The mixture was heated to 75° C. to 80° C. and a solution formed with a viscous oil present. The oil dispersed after ˜1 hour to form an amber solution. Reaction progress was monitored by HPLC. After heating for 4 to 6 hours, the reaction mixture was typically 75% to 85% desired product with 2 other impurities present. The reaction mixture was also heated for over 14 hours in one lot and no negative impact was observed. After heating for 6 hours, activated carbon (−10 wt % of starting material) was added to the solution and stirred for 10 minutes while hot. Concentrated sulfuric acid (3.3 mL) was then charged to the reaction mixture (Note: if the sulfuric acid solution is allowed to cool, the product sulfate salt will begin to precipitate which is undesirable). The reaction mixture was filtered through a glass fiber filter and the charcoal was washed with a small amount of water, RO. The filtrate was concentrated on a Buchi rotoevaporator at a bath temperature of 60° C. to 70° C. to dryness to remove the HCl. The resulting oil was then co-evaporated with 4×50 mL of water, RO to further remove residual HCl. Solids may or may not precipitate during this operation depending on the internal temperature of the concentrate. Solids do re-dissolve when heated above 65° C. The dry residue was dissolved in water, RO (60 mL, 3 volumes of starting material) and ethanol (240 mL, 12 volumes of starting material) at reflux (˜70° C.) to form a yellow solution after 5 to 10 minutes. The solution was then cooled to −15° C. to precipitate the product. The slurry was stirred at −15° C. for 1 to 2 hours. The product was filtered on a filter funnel through a polypad and washed with cold ethanol (3×40 mL). The product was then pulled dry on the filter funnel. The product was placed on a drying tray and dried in a vacuum oven at 30° C. and at <10 mm Hg overnight to remove all residual solvents. Yield: 14.7 g (0.0410 mol, 70%, white solid). HPLC: 98.5% chemical purity. Chiral HPLC: 100% ee.

### Example 6

### Preparation of L-4-Chlorokynurenine from L-4-Chlorokynurenine Sulfate Monohydrate using Caustic Soda

L-4-chlorokynurenine sulfate monohydrate was dissolved in water, RO and heated to ˜60° C. Caustic soda (2 equiv) was then added to the solution and the free base began to re-precipitate immediately. The slurry was cooled to 5° C. and stirred for 1 hour. The pH of this slurry was ˜5. The product free base was recovered in a 54% yield after filtration and drying under reduced pressure. To increase the yield of the free base, three different organic anti-solvents (acetonitrile, THF, and acetone) were added to the warm solutions, before addition of caustic soda, in three different trials. The THF trial resulted in no precipitation of the free base and was discarded. The acetonitrile and acetone trials both formed even slurries. The product from the acetonitrile trial was isolated. Yield: 65%. HPLC: 99.7% chemical purity. The product from the acetone trial was isolated. Yield: 110% (due to some precipitation of inorganic salts along with the desired product). HPLC: 99.8% chemical purity.

### Example 7

### Preparation of L-4-Chlorokynurenine from L-4-Chlorokynurenine Sulfate Monohydrate using Amberlite Resin

L-4-chlorokynurenine sulfate monohydrate was dissolved in methanol, AR (analytical reagent) and stirred for approximately 15 to 20 minutes until a clear solution formed. Amberlite resin (FPA 53, 5 vol) was then charged, and an off-white suspension formed. The suspension was stirred for a minimum of 12 hours at ambient temperature. The reaction mixture was filtered through a 100 μm glass-fiber filter paper or filter cloth, and the resin was reslurried with methanol AR (12 vol), filtered, and washed with methanol AR (approximately 1 vol). The combined filtrates were concentrated at a jacket temperature of 30° C. to 35° C. to a minimum stirrable volume, which resulted in a thick slurry. The resulting off-white suspension was then diluted with ethyl acetate (12 vol). The product was collected using a sub-micron centrifuge bag and washed with 2 vol of ethyl acetate. The product was then spun-dry on the centrifuge. The product was placed on a drying tray and dried in a vacuum oven at 35° C. (<10 mm Hg) overnight to remove all residual solvents. Yield: 2,011 g (50% overall yield, two steps), light-yellow solid. HPLC: 99.6% chemical purity. Chiral HPLC: 100% ee.

What is claimed is:

1. A method of preparing a chiral tryptophan compound or a pharmaceutically acceptable salt, polymorph, hydrate, solvate, tautomer, or stereoisomer thereof of Formula IVb: 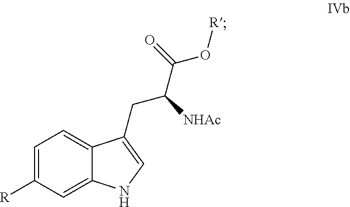
wherein R is halogen; and
wherein R′ is selected from the group consisting of alkyl and substituted alkyl;
the method comprising:
a) enantioselectively hydrogenating an unsaturated tryptophan compound of Formula IIIb with a chiral catalyst to afford the chiral tryptophan compound of Formula IVb:

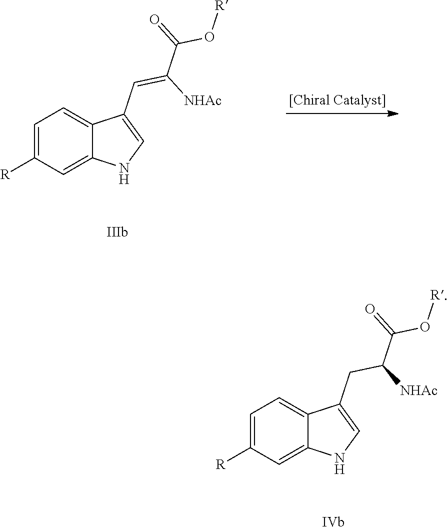

2. The method of claim 1, wherein R′ is an alkyl selected from the group consisting of methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl.

3. The method of claim 1, wherein R is a halogen selected from the group consisting of fluoro, chloro, bromo, and iodo.

4. The method of claim 1, wherein the chiral catalyst comprises a chiral rhodium catalyst.

5. The method of claim 4, wherein the chiral rhodium catalyst comprises a chiral phosphine ligand and rhodium.

6. The method of claim 5, wherein the chiral phosphine ligand is an enantiomer of DuanPhos or an enantiomer of DuPhos.

7. The method of claim 5, wherein the chiral phosphine ligand is an enantiomer of DuanPhos.

8. The method of claim 5, wherein the chiral tryptophan compound of Formula IVb is (S)-ethyl 2-acetamido-3-(6-chloro-1H-indol-3-yl)propanoate.

9. A method of preparing a compound of Formula I or a pharmaceutically acceptable salt, polymorph, hydrate, solvate, tautomer, or stereoisomer thereof: 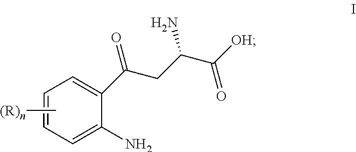
wherein each R is independently selected from the group consisting of hydrogen, halogen, alkyl, and substituted alkyl; and
wherein n=0-4;
the method comprising:
a) coupling an indole aldehyde compound of Formula II with an acetamidomalonate compound of Formula IIa in the presence of a suitable anhydride compound in a suitable solvent to afford an unsaturated tryptophan compound of Formula IIIa: 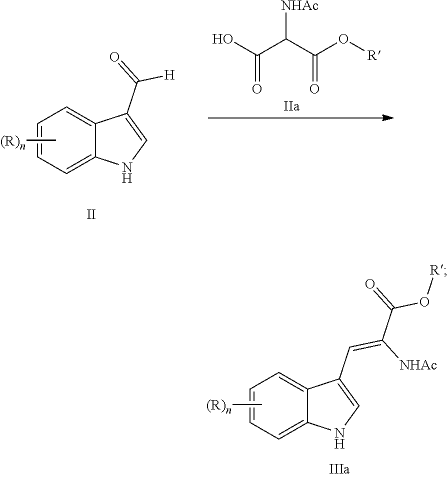
wherein R′ is selected from the group consisting of alkyl and substituted alkyl;
b) enantioselectively hydrogenating the unsaturated tryptophan compound of Formula IIIa with a chiral catalyst to afford the chiral tryptophan compound of Formula IVa: 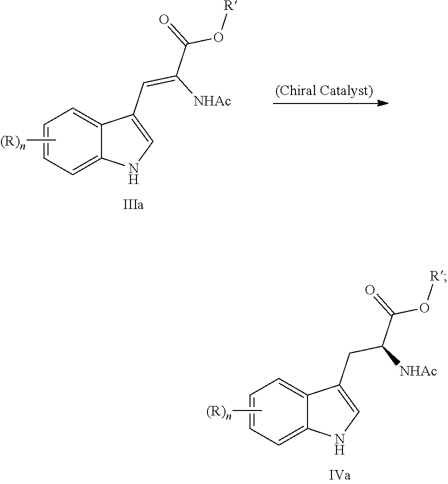
c) oxidizing the chiral tryptophan compound of Formula IVa with an oxidizing agent to afford a compound of Formula V: 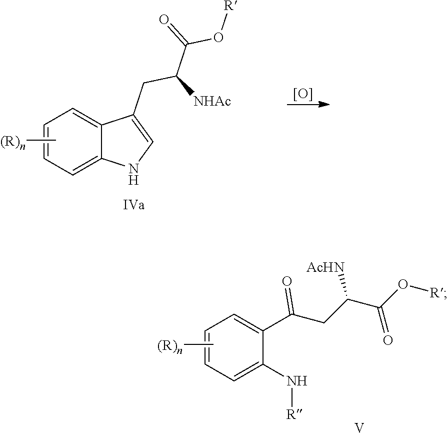
wherein R″ is selected from the group consisting of formyl and hydrogen; and
d) deprotecting the compound of Formula V to afford the compound of Formula I:

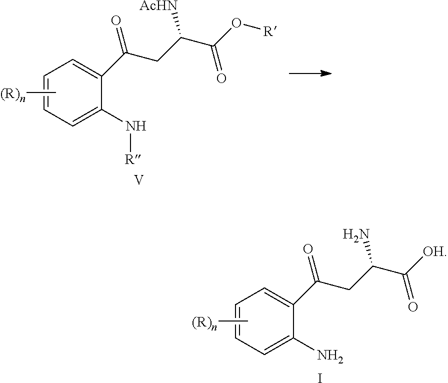

10. The method of claim 9, wherein R′ is an alkyl selected from the group consisting of methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl.

11. The method of claim 9, wherein n=1 and R is a halogen selected from the group consisting of fluoro, chloro, bromo, and iodo.

12. The method of claim 9, wherein in step a) the suitable anhydride compound is acetic anhydride and the suitable solvent is pyridine.

13. The method of claim 9, wherein the chiral catalyst comprises a chiral rhodium catalyst.

14. The method of claim 9, wherein the chiral rhodium catalyst comprises a chiral phosphine ligand and rhodium.

15. The method of claim 14, wherein the chiral phosphine ligand is an enantiomer of DuPhos or DuanPhos.

16. The method of claim 14, wherein the chiral phosphine ligand is an enantiomer of DuanPhos.

17. The method of claim 9, wherein the oxidizing agent is selected from the group consisting of m-chloroperoxybenzoic acid, potassium peroxysulfate, sodium periodate, ozone, superoxide, peracetic acid, and RuCl₃/sodium periodate.

18. The method of claim 17, wherein the oxidizing agent is m-chloroperoxybenzoic acid.

19. The method of claim 9, wherein the deprotecting comprises heating the compound of Formula V in the presence of HCl.

20. The method of claim 9, wherein the deprotecting comprises heating the compound of Formula V in the presence of HCl followed by addition of sulfuric acid and isolation of the compound of Formula I as a sulfate monohydrate salt.

21. The method of claim 20, wherein the sulfate monohydrate salt is reacted with sodium hydroxide to afford the compound of Formula I as a free base.

22. The method of claim 20, wherein the sulfate monohydrate salt is reacted with Amberlite resin to afford the compound of Formula I as a free base.

23. The method of claim 9, wherein the compound of Formula I is L-4-chlorokynurenine.

24. A method of preparing the compound: 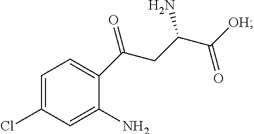 [SMILES: Nc1cc(Cl)ccc1C(=O)C[C@H](N)C(=O)O]
the method comprising:
a) coupling 6-chloroindole-3-carboxaldehyde with ethyl acetamidomalonate in the presence of acetic anhydride in pyridine solvent to afford ethyl Z-α-acetamido-6-chloroindole-3-acrylate: 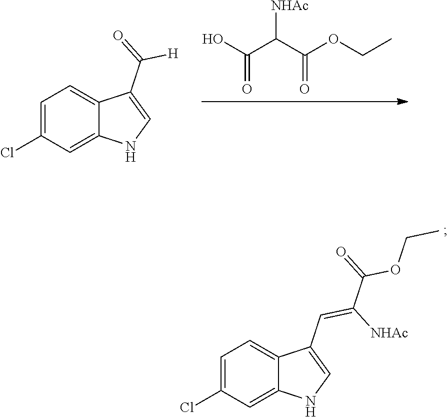 [SMILES: O=Cc1c[nH]c2cc(Cl)ccc12 ; CCOC(=O)C(NC(C)=O)C(=O)O ; CCOC(=O)/C(=C/c1c[nH]c2cc(Cl)ccc12)NC(C)=O]
b) enantioselectively hydrogenating ethyl Z-α-acetamido-6-chloroindole-3-acrylate with [(S,S′,R,R′-DuanPhos)Rh(COD)][BF₄] to afford (S)-ethyl 2-acetamido-3-(6-chloro-1H-indol-3-yl)propanoate: 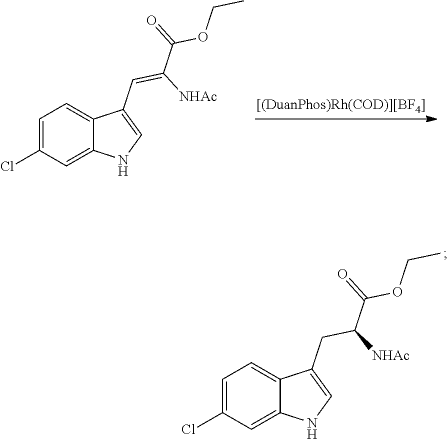 [SMILES: CCOC(=O)/C(=C/c1c[nH]c2cc(Cl)ccc12)NC(C)=O ; CCOC(=O)[C@H](Cc1c[nH]c2cc(Cl)ccc12)NC(C)=O]
c) oxidizing (S)-ethyl 2-acetamido-3-(6-chloro-1H-indol-3-yl)propanoate with m-chloroperoxybenzoic acid (MCPBA) to afford ethyl (2S)-2(acetamido)-4-(2-carbonylamino-4-chlorophenyl)-4-oxobutanoate: 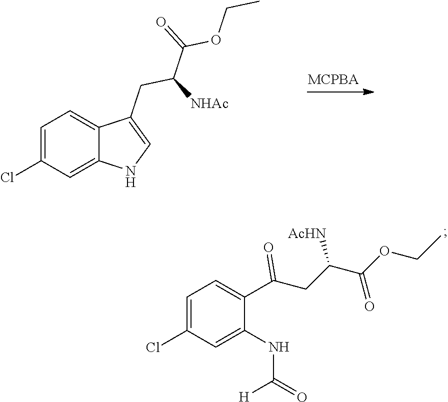 [SMILES: CCOC(=O)[C@H](Cc1c[nH]c2cc(Cl)ccc12)NC(C)=O ; CCOC(=O)[C@H](CC(=O)c1ccc(Cl)cc1NC=O)NC(C)=O]
and
d) deprotecting ethyl (2S)-2(acetamido)-4-(2-carbonylamino-4-chlorophenyl)-4-oxobutanoate to afford L-4-chlorokynurenine:

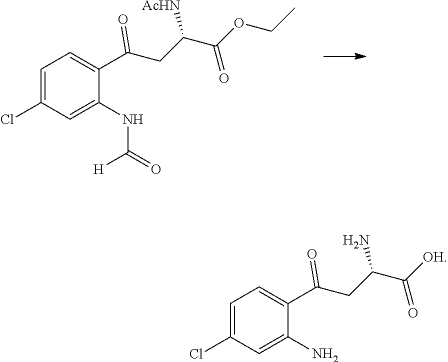 [SMILES: CCOC(=O)[C@H](CC(=O)c1ccc(Cl)cc1NC=O)NC(C)=O ; Nc1cc(Cl)ccc1C(=O)C[C@H](N)C(=O)O]

25. The method of claim 24, wherein the deprotecting step comprises generating L-4-chlorokynurenine sulfate monohydrate.

26. The method of claim 25, wherein the sulfate salt of the L-4-chlorokynurenine sulfate monohydrate is removed to afford L-4-chlorokynurenine.

27. A compound having the structure:

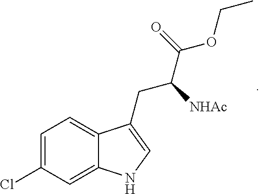

28. A compound having the structure:

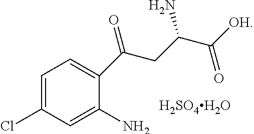

29. A pharmaceutical composition comprising the compound of claim 28.

30. The composition of claim 29 comprising the compound in at least about 95% chemical purity and at least about 95% ee.

31. The pharmaceutical composition of claim 29, wherein the compound is prepared by the method of claim 24.
